(19) United States
(12) Patent Application Publication
(10) Pub. No.: US 2024/0252566 A1
(43) Pub. Date: Aug. 1, 2024

(54) A WATER-SOLUBLE GLASS RUMEN BOLUS

(71) Applicant: BIMEDA ANIMAL HEALTH LIMITED, Carrickmines, Dublin (IE)

(72) Inventors: Daniel Peter TIERNEY, Blackrock (IE); Donal Thomas Martin TIERNEY, Blessington (IE)

(73) Assignee: BIMEDA ANIMAL HEALTH LIMITED, Carrickmines, Dublin (IE)

(21) Appl. No.: 18/560,449

(22) PCT Filed: May 27, 2021
(86) PCT No.: PCT/EP2021/064288

§ 371 Date: Nov. 13, 2023

(30) Foreign Application Priority Data
May 14, 2021 (GB) 2106921.6

(51) Int. Cl. A61K 36/04; A61K 9/00; A61K 31/02; A61K 47/12

(52) U.S. Cl. CPC A61K 36/04; A61K 9/0068; A61K 31/02; A61K 47/12

(57) ABSTRACT

A water-soluble glass bolus article in a form suitable for oral administration to a ruminant animal, the article comprising carboxylate glass and an organic active agent. Metal carboxylates such as acetates, propanoates and butanoates can be melted to a glass and have a lower melting temperature than phosphate-based glass materials, allowing thermally sensitive active agents to be formulated into a glass bolus article. The carboxylate glass is formed from one or more metal carboxylates having a melting point of less than 300° C. in which the anion is selected from one or more acetates, propanoates, or a mixture of carboxylate anions. The metal may be selected from sodium, potassium, calcium and zinc.

### FIELD OF THE INVENTION

The present invention relates to a water-soluble glass rumen bolus, a method of manufacturing a water soluble glass rumen bolus, and method of administering a temperature sensitive active ingredient in a ruminant animal.

### BACKGROUND TO THE INVENTION

Water soluble glass bolus articles are currently employed to deliver inorganic elements such as copper, selenium, cobalt, zinc, iodine and magnesium to ruminant animals (GB2163346). The bolus article is formed by melting glass forming materials and one or more elements to form a liquid glass and then cooling and shaping the liquid glass into a shaped article suitable for administering orally to a ruminant animal. The glass forming materials comprise a phosphate such as sodium metaphosphate or sodium hexametaphosphate, an alkali metal such as Na₂O or K₂O, and a metal oxide such as CaO or MgO. The formed article is a water-soluble glass that is administered to the reticulo rumen of the animal, where it slowly dissolves over a period of months to release the inorganic elements. As the inorganic elements have a high melting point and are only sensitive to very high temperatures, this allows the use of phosphate-based glass forming materials which melt at very high temperatures without any damage to the inorganic elements. However, this technology is only applicable to delivering inorganic active agents that have very high melting points and is not suitable for use with more temperature sensitive active agents such as vitamins and other organic compounds.

It is an objective of the invention to overcome at least one of the above-referenced problems.

### SUMMARY OF THE INVENTION

The objective is met by the provision of a glass bolus in which the glass forming phosphate-based materials are replaced with low-temperature glass forming materials such as metal carboxylates that have a lower melting temperature compared with phosphate-based materials (e.g. less than 300° C.). This allows organic active agents to be formulated into a water-soluble glass bolus without loss of activity. The Applicant has discovered that a suitable form of glass bolus article is a sintered glass article, which may be formed by melting and cooling the metal carboxylates to form a solid glass, milling the solid glass to form particles, mixing the organic active agent with the particles of glass, pressing the mixture into a bolus article of required dimensions, and then sintering the pressed article to form a water-soluble glass bolus article suitable for lodging in the reticulo rumen of the animal. Sintering temperatures suitable for forming the glass bolus article are also relatively low compared with melting temperatures required for phosphate-based materials and are amendable for use with many organic active agents.

In a first aspect, the invention provides a water-soluble glass bolus article in a form suitable for oral administration to an animal, typically a ruminant animal, the article comprising:
carboxylate glass; and
an organic active agent.

In a second aspect, the invention provides a water-soluble glass bolus article in a form suitable for oral administration to an animal, typically a ruminant animal, the article comprising:
carboxylate glass; and
bromoform.

In a third aspect, the invention provides a method of forming a water-soluble glass bolus article comprising the steps of:
mixing one or more metal carboxylates and an organic active agent to provide a mixture;
melting the mixture to form a liquid carboxylate glass containing organic active agent; and
casting the liquid carboxylate glass containing organic active agent into a solid water soluble glass bolus article.

In a fourth aspect, the invention provides a method of forming a water-soluble glass bolus article comprising the steps of:
melting a metal carboxylate to form a liquid carboxylate glass;
allowing the liquid carboxylate to cool to provide a solid carboxylate glass;
milling the solid carboxylate glass to provide carboxylate glass particles;
mixing the carboxylate glass particles with an organic active agent and optionally a binder;
forming the mixture into a bolus article; and
sintering the bolus article to form a sintered water-soluble glass bolus article.

In a fifth aspect, the invention provides a method of reducing methane production in an animal, especially a ruminant animal, the method comprising a step of administering a water-soluble glass bolus article to a stomach of the animal, in which the organic active agent is bromoform. The animal may be a monogastric animal or a ruminant animal—in the latter case the water-soluble glass bolus article is administered to the reticulo rumen of the animal.

Other aspects and preferred embodiments of the invention are defined and described in the other claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein the term “animal” refers to a monogastric animal (for example a horse) or a ruminant animal (for example a bovine, ovine or cuprine animal). In a preferred embodiment, the animal is bovine.

As used herein, the term “water-soluble glass bolus article” refers to a solid article suitable for administration to a stomach of an animal, especially a ruminant animal, especially the reticulo rumen of a ruminant animal, that comprises carboxylate glass (or another low-temperature glass forming material) and an active agent, typically an organic active agent. The bolus article may be administered by a bolus applicator the details of which are known to a person skilled in the art. Metal carboxylates such as acetates, propanoates and butanoates can be melted to a glass and have a lower melting temperature that phosphate-based glass materials, allowing thermally sensitive active agents to be formulated into a glass bolus. The metal carboxylate (or mixture of metal carboxylates) employed generally have a melting point of less than 300° C., 250° C. or 200° C. In one embodiment, the glass forming carboxylate(s) are mixed with the active agent, melted to a liquid glass, and then cast into a glass bolus article. In any embodiment, the article is sintered. This means it is formed from carboxylate glass particles that are sintered to form the solid article. In one embodiment, the glass if formed without the active agent, and then milled into particles and the active agent (and optionally binder or other agents) is added, and the mixture is pressed into the desired shape and sintered to form the glass bolus article. In any embodiment, the sintered article comprises a binder. In any embodiment, the glass bolus article has a weight of 10 to 200 g, 50 to 200 g, 10 to 50 g, 50 to 150 g, 75 to 125 g, and ideally about 90 to 110 g. In any embodiment, the glass bolus article contains 1-20%, 1-5%, 5-15%, 5-10%, 10-15%, 15-20% organic active agent by weight. In any embodiment, the glass bolus article is configured to disintegrate in a stomach, for example a reticulo rumen, of the animal over a period of at least 1, 2, 3, 4, 5, 10, 15, 20, 25, 30, 35, 40, 45, 50, 55 or 60 days, for example 1 to 150 days, 10 to 120 days, 30 to 90 days, 40 to 80 days, 50 to 70 days, 1 to 5 days, 1 to 10 days, 10 to 20 days, 20 to 30 days, 30 to 40 days, 40 to 60 days, 50 to 70 days, or 50 to 60 days. In any embodiment, the glass bolus article is configured to release organic active agent in a stomach, for example a reticulo rumen of the animal at a rate of 10 to 500 mg, 10 to 200 mg, 50 to 150 mg, 80 to 120 mg, per day. In any embodiment, the glass bolus article has a specific gravity of 1 to 7, 1 to 6, 1 to 5, 1 to 4, 2 to 5, 2 to 4 g/cm³. In any embodiment, the glass bolus article has a surface area of 10 to 150, 10 to 100, 10 to 50, or 50 to 100 cm². In any embodiment, the glass bolus article has a curved diameter of 10 to 50, 10 to 25, 25-50 mm.

As used herein, the term “carboxylate glass” refers to a glass formed when one or a mixture of metal carboxylates are heated to their melting temperature or above and allowed cool. In any embodiment, the carboxylate glass is selected from an acetate glass, a propanoate glass, a butanoate glass, and a mixed anion glass. The mixed anion glass may include carboxylates selected from an acetate, a propanoate, a butanoate and an octanoate. In any embodiment, the carboxylate glass comprises (or is formed from) a mixture of metal carboxylates, for example a mixture of metal acetates or a mixture of metal propanoates. Examples of carboxylates glasses are provided in Table 1 below. In any embodiment, the carboxylate glass comprises (or is formed from) at least 2, 3 or 4 metal carboxylates. In any embodiment, the cation of the metal carboxylate is selected from sodium, potassium, calcium and zinc, although other metals may be employed.

TABLE 1

| | Liquidus | Estimated | |
|---|---|---|---|
| | temperature | duration of | Density |
| Glass composition ratio | (° C.) | glass | g/cm3 |
| Acetate glass 1.1.1 | 140 | 2 hours | 1.48 |
| Sodium acetate | | | |
| Potassium acetate | | | |
| Calcium acetate | | | |
| Propanoate glass 1.1 | 180 | 8 hours | 1.32 |
| Sodium propanoate | | | |
| Calcium propanoate | | | |
| Mixed anion glass 1.2.2 | 160 | 2 weeks | 1.18 |
| Sodium butanoate | | | |
| Calcium propanoate | | | |
| Sodium octanoate | | | |

As used herein, the term “organic active agent” refers to an active agent that is thermally unstable and would inactivate or degrade at the high temperatures employed to may phosphate-based water-soluble glass boluses. Examples include vitamins (for example Vitamin A, Vitamin D3), amino acids (both natural amino acids and variants such as D and L amino acids, and amino acid analogues), peptides, proteins, carbohydrates, lipids, antibiotics, and bioactive agents such as plant extracts. In any embodiment, the organic active agent is thermally unstable and loses some or all of its activity when heated to a temperature above 200° ° C.

In any embodiment, the organic active agent is bromoform. As used herein the term “bromoform” refers to a halogenated organic solvent obtainable from certain seaweed species, including species of Asparagopsis such as for example Asparagopsis taxiformis and/or Asparagopsis armata. In any embodiment, the bromoform is provided as a seaweed extract enriched in bromoform. The term “enriched in bromoform” means that bromoform constitutes at least 50% by weight of the seaweed extract. In any embodiment, bromoform constitutes at least 60%, 70% or 80% by weight of the seaweed extract. Seaweed extracts containing 10-50% bromoform may also be employed. In any embodiment, the bromoform is a methanolic extract of seaweed. Methods of obtaining bromoform methanolic extracts from seaweed are described in WO2020124167 (methanolic extract method pages 36-37 and Table 2). In any embodiment, the bromoform is an oil extract of seaweed. Methods of obtaining bromoform oil extracts from seaweed are described in WO2020113279 (oil extract method of Example 1 pages 47 to 51). Additional methods of extracting bromoform from seaweed are described in WO2005/015983; U.S. Pat. No. 6,346,252; Algal Research, Volume 51, Pages 102065 (2020); J. Appl. Phycol., 28 (2016), pp. 3117-3126; Limnology and Oceanography, Issue: 5, Volume: 44, Pages 1348-1352. Jul. 13, 1999; Tetrahedron Letters, Issue: 7, Volume: 16, Pages: 473-476. 1975; Heliyon, Volume 4, Issue 11, November 2018, e00957; and Mar Ecol Prog Ser 306:87-101 (2006a).

The invention also provides a method of forming a water-soluble glass bolus article. In one embodiment, the glass forming carboxylate(s) are mixed with the active agent, melted to a liquid glass, and then cast into a glass bolus article. In another embodiment, the method comprises melting one or more metal carboxylates to form a liquid carboxylate glass, cooling the liquid carboxylate to provide a solid carboxylate glass, milling the solid carboxylate glass to provide carboxylate glass particles, mixing the carboxylate glass particles with an organic active agent, and pressing the mixture to form a bolus article and sintering the mixture to form a sintered water-soluble glass bolus article. In any embodiment, the melting step is performed at a temperature of 50 to 300° C., 100 to 300° C., 100 to 250° C., 100 to 200° C., 150 to 300° C., 200 to 300° C., 150 to 250° C. or 200 to 250° C. In any embodiment, the sintering step is performed at a temperature of 20 to 250° C., 50 to 250° C., 100 to 250° C., 150 to 250° C., 50 to 200° C., 100 to 200° C. or 150 to 200° C. In any embodiment, the sintering step comprises an annealing step. In any embodiment, the annealing step is performed at a temperature of 20 to 200° C., 50 to 200° C., 100 to 200° C., 150 to 200° C., 50 to 1500° C., or 150 to 200° C. In any embodiment, the milling step is configured to provide carboxylate glass particles having an average particle size of 0.1 to 1 mm, 0.5 to 1 mm, 0.1 to 0.5 mm. In any embodiment, the mixing step comprises adding a binder to the carboxylate glass particles and organic active agent. In any embodiment, the mixture of carboxylate glass particles, organic active agent and optionally binder is granulated prior to the sintering step. Sintering or frittage is the process of compacting and forming a solid mass of material by heat and/or pressure without melting it to the point of liquefaction. It is described in German et al. (Encyclopedia of Materials: Science and Technology, 2001 and Sintering: from Empirical Observations to Scientific Principles, 2014.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

A mixture of 1:1:1 sodium acetate, potassium acetate and calcium acetate is mixed together and heated to 140° C. until the mixture liquifies. The liquid glass is allowed to cool and is then milled and screened to an average particle size of 0.1 to 1 mm. Lyophilised seaweed extract is prepared according to the method of Example 1 of WO2020113279. The extract contains about 60 bromoform by weight. The lyophilized extract is added to the particulate glass in a weight ratio of 1 part seaweed extract to 6 parts particulate glass, mixed with any binding agent if applicable and pressed into a bolus of required dimensions. The pressed boluses are then sintered at a pre-determined temperature and annealed at a pre-determined temperature (obtained through analysis of the glass by differential scanning calorimetry).

### Example 2

The method of Example 1 is repeated except that the glass is formed from 1:1 mixture of sodium propanoate and calcium propanoate heated to 180° C. until the mixture liquifies.

### Example 3

The method of Example 1 is repeated except that the glass is formed from a mixture of 1:2:2 Sodium butanoate, Calcium propanoate and Sodium octanoate heated to 160° C. until the mixture liquifies.

### Example 4

The method of Example 1 is repeated except that the bromoform extract is replaced by a calcium salt of the methionine hydroxy analogue (HMTBA-Ca,C5H10O3S2Ca).

### Example 5

15 g of lyophilised seaweed extract is prepared according to the method of Example 1 of WO2020113279. The extract contains about 10 g bromoform. A 2:2:2:1 mixture of sodium acetate, potassium acetate, calcium acetate and lyophilised seaweed extract is mixed together and heated to 140° C. until the mixture liquifies. The liquid glass is cast into glass articles having a diameter of 1.5 cm, length of 4 cm, and weight of 27 g.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A water-soluble glass bolus article in a form suitable for oral administration to a ruminant animal, the article comprising:
carboxylate glass; and
an organic active agent.

2. A water-soluble glass bolus article according to claim 1 in which the carboxylate glass is formed from a metal carboxylates having a melting point of less than 300° C.

3. A water-soluble glass bolus article according to claim 2 formed from a mixture of metal acetates.

4. A water-soluble glass bolus article according to claim 2 formed from a mixture of metal propanoates.

5. A water-soluble glass bolus article according to claim 2 in which the plurality of metal carboxylates comprises different anions.

6. A water-soluble glass bolus article according to any preceding claim comprising 1-20% organic active agent by weight.

7. A water-soluble glass bolus article according to any preceding claim in which the organic active agent is selected from a vitamin, antibiotic, and plant extract.

8. A water-soluble glass bolus article according to any preceding claim in which the plant extract is a seaweed extract enriched in bromoform.

9. A water-soluble glass bolus article according to any preceding claim in which the glass bolus article has a specific gravity of 1 to 7 g/cm3.

10. A water-soluble glass bolus article according to any preceding claim in which the glass bolus article has a specific gravity of 2 to 4 g/cm3.

11. A method of forming a water-soluble glass bolus article comprising the steps of:
melting one or more metal carboxylates at a temperature of less than 300° ° C. to form a liquid carboxylate glass;
allowing the liquid carboxylate to cool to provide a solid carboxylate glass;
milling the solid carboxylate glass to provide carboxylate glass particles;
mixing the carboxylate glass particles with an organic active agent;
pressing the mixture to form a bolus article; and
sintering the bolus article to form a sintered water-soluble glass bolus article.

12. A water soluble glass bolus article according to any of claims 1 to 9, or formed by a process of claim 10 or 11, for use in a method of administering an organic active agent to an animal by sustained release, the method comprising orally administering the water soluble glass bolus article to a stomach of the animal wherein the organic active agent is released from the water soluble glass bolus article in the stomach of the animal over a sustained period of time.

13. A water soluble glass bolus article according to any of claims 1 to 9, or formed by a process of claim 10 or 11, for use of claim 12, in which the animal is a ruminant animal, wherein the water soluble glass bolus article is administered to the reticulo rumen of the animal.

14. A water soluble glass bolus article according to any of claims 1 to 9, or formed by a process of claim 10 or 11, for use of claim 12 or 13, in which the organic active agent is released from the water soluble glass bolus article in the animal over a period of 1-120 days.

15. A water soluble glass bolus article according to any of claims 1 to 9, or formed by a process of claim 10 or 11, for use of any of claims 12 to 14, in which the organic active agent is a seaweed extract enriched in bromoform.

16. A water soluble glass bolus article according to any of claims 1 to 9, or formed by a process of claim 10 or 11, for use of claim 15, in which the water soluble glass bolus article comprises 5-15 g bromoform, and is configured to release 10-200 mg bromoform per day in the stomach of the animal.

17. A water soluble glass bolus article according to claim 8, for use in a method of reducing methane production in ruminant animals, the method comprising a step of administering the water-soluble glass bolus article to the reticulo rumen of the animal, in which the organic active agent is bromoform.
